(19) United States
(12) Patent Application Publication
(10) Pub. No.: US 2015/0291992 A1
(43) Pub. Date: Oct. 15, 2015

(54) METHOD AND APPARATUS FOR DETERMINING MICROORGANISMS IN A WATER SAMPLE

(71) Applicants: SAUDI ARABIAN OIL COMPANY, DHAHRAN (SA); DANISH TECHNOLOGICAL INSTITUTE, Aarhus C (DK)

(72) Inventors: Mohammed A. AL-MONIEE, Dhahran (SA); Lone TANG, Hoejbjerg (DK); Susanne JUHLER, Aarhus C. (DK); Niels Vinther VOIGT, Aarhus N. (DK); Peter Frank SANDERS, Devon (GB)

(21) Appl. No.: 14/621,984

(22) Filed: Feb. 13, 2015

Related U.S. Application Data
(60) Provisional application No. 61/977,330, filed on Apr. 9, 2014.

(51) Int. Cl. C12Q 1/04; G01N 33/18

(52) U.S. Cl. CPC C12Q 1/04; G01N 33/18

(57) ABSTRACT

The invention relates to an apparatus and method for determining presence of microorganisms in, e.g., sea water or other water samples. Means are provided for taking a sample of the water to be tested, moving it through a series of reservoirs, and combining it with an indicator, such as a dye, which binds to DNA of any microorganisms in the sample. Any binding of indicator is measured by, e.g., a spectrofluorometer, or other device suitable for measuring the indicator.

### CROSS-REFERENCE TO RELATED APPLICATIONS

The present application claims priority on U.S. provisional application No. 61/977,330 filed Apr. 9, 2014.

### FIELD OF THE INVENTION

The invention relates to methods and apparatus useful in continuous, automated, real time determination of microorganisms in flowing water systems, such as systems involving seawater, using a biosensor and DNA staining technology.

### BACKGROUND AND PRIOR ART

In any system involving flowing water, the presence of microorganisms in the water can influence the system in negative ways. Examples of such problems include microbially influenced corrosion of instruments, clogging of the system, or reservoirs, biofouling and so forth. See, e.g., U.S. Pat. No. 8,525,130, incorporated by reference, which discusses problems caused by biofouling in seawater desalination plants, and efforts to detect and to analyze, e.g., biofilms which grow on the apparatus of the plants.

While there are many methodologies known for determining presence of microorganisms using biosensors, there is limited information available in the applicability of these methods to fluids, such as natural waters, recreational waters, seawater, and so forth. See in this regard, U.S. Pat. No. 8,206,946, also incorporated by reference.

U.S. Pat. No. 6,787,302, the disclosure of which is incorporated by reference, teaches the use of a commercially available dye “SYTO16,” for determining viable cells in a fluid sample. Also see U.S. Pat. No. 8,206,946, Published U.S. Application 20040191859, and PCT Application WO 1995 0191859, which disclose the use of fluorescent dyes for determining microorganisms. All are incorporated by reference.

None of these references teach or disclose methods and/or apparatus useful in real time analysis systems, which can be used for automated and continuous monitoring of the presence of microorganisms in water samples, such as seawater.

The invention which is disclosed infra is directed to apparatus and methods which address the issues set forth supra.

### SUMMARY OF THE INVENTION

The invention relates to methods and apparatus useful in automated monitoring microorganisms in flowing water systems, such as seawater, on a continuous basis. Further, the invention comprises incorporating DNA staining technology into an autonomous microbe sensor, thus enabling detection of microorganisms via staining DNA, which is ubiquitous in microorganisms.

### BRIEF DESCRIPTION OF THE FIGURES

FIG. 1 shows an embodiment of the invention.

FIG. 2 shows results from one experiment carried out in accordance with the invention.

FIGS. 3a and 3b show further results.

FIG. 4 shows yet further results.

### DETAILED DESCRIPTION OF PREFERRED EMBODIMENTS

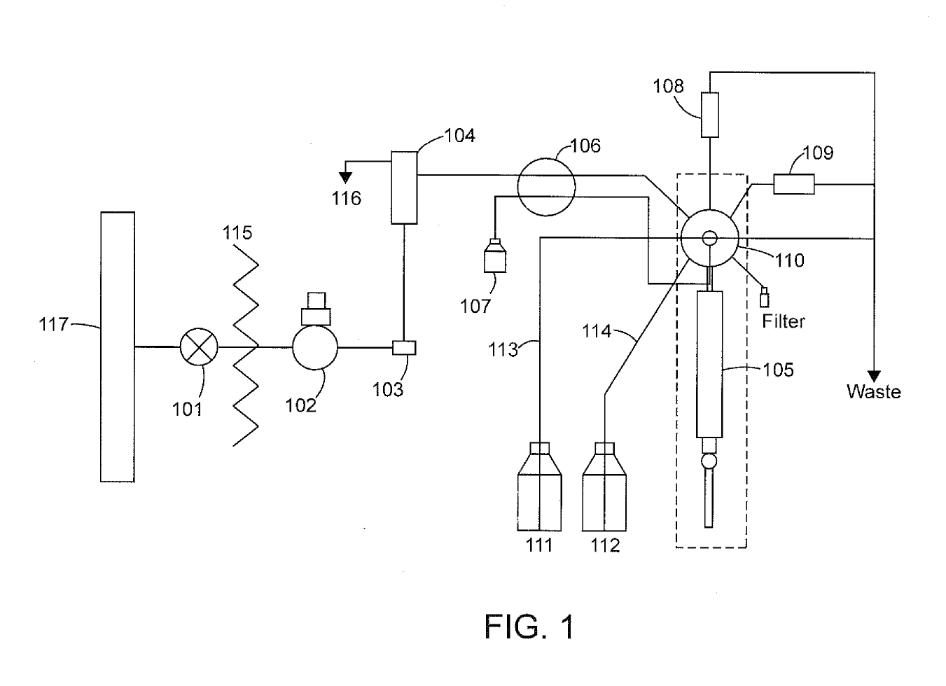

One embodiment of the invention is shown in FIG. 1. Referring thereto a pipeline valve “101” is depicted, connected to pipeline 117. This serves as the connector interphase between the biosensor, discussed infra, and the seawater injecting side stream. Also depicted is a pressure regulator 102, which regulates the pressure of water injected into the biosensor, and ensures stable flow into a sampling chamber. As developed infra, the pressure of the water should be below 10 bar.

Delivery of the water in a stable, regular flow is also facilitated by a tubing means with a restrictor element “103.” The water flows through the tubing means into sampling chamber “104.” This chamber has a constant overflow, which ensures constant availability of fresh samples. Also shown is an automated syringe “105,” which is calibrated to take precise sample volumes (e.g., 5 ml), as facilitated by a two port injection valve “106” and distribution valve “110”. The injection valve 106 removes water from the sample chamber, and also from indicator reservoir “107.” When sample and DNA stain are being mixed, automated syringe “105” draws the liquids back and forth, after which they move into mixing chamber “108,” to further ensure uniform mixing. More generally, however, the syringe is used to take samples of, e.g., liquid from the sampling chamber, the indicator reservoir, reservoirs 111 and 112, discussed infra, and air from the air filter, not shown herein. Liquid or air can then be moved toward sampling chamber, indicator reservoir, the mixing chamber discussed infra, the flow cell, also discussed infra, and the water reservoir. Positions of valve 110 and injection valve 106 control the taking and distribution of sample. Also shown is flow cell “109,” which may be viewed as a flow through cuvette, a first reservoir “111” containing, e.g., distilled water, and a second reservoir “112,” which contains a cleaning agent, each of which are provided with means “113” and “114” for drawing the respective materials into the working apparatus are also shown. In a preferred embodiment, distribution valve 110 is motorized. As is shown in the embodiment of FIG. 1, it has a plurality of ports (in this embodiment, 8, each of which is represented by a lead line), and it controls from where the syringe draws liquid or air, and to where the syringe content is distributed when the syringe is emptied. What is also shown herein is a portion of an enclosure means 115 used to protect the apparatus from the environment. The sensor itself is not shown in this figure. After measurement, of sample waste material is transferred via means 116 to, e.g., a waste reservoir, which is not shown.

In operation, the apparatus is turned to “on” mode automatically or manually. When this is done automatically, a timer, a computer control, inter or intranet connections, and so forth, may be used. When an automated timer is used, this is configured so as to turn on a computer using standard or customized software, or by programming a computer which is part of the biosensor, or by remote control, via interior intranet connection. Automated timer means utilize the least energy but other systems may be used.

As can be seen from the description supra, the apparatus of the invention uses a dispensing system to transport and mix fluids (e.g., water samples, dye solution, cleansing agents, and rinsing water). In the depicted embodiment, syringes are used, but the skilled artisan will see the possibility of other modes for dispensing and mixing.

In operation, a sample of water, e.g., seawater is removed from the sampling chamber, and a precise amount, as discussed infra, is mixed with an indicator, such as a DNA binding dye, in a predetermined, but variable ratio. The ratio depends on many factors including the nature of the indicator, the salinity of the liquid being tested, and other factors. In the examples, SYBR® Green I ((N′,N′-dimethyl-N-[4-[(E)-(3-methyl-1,3-benzothiazol-2-yildene)methyl]-1-phenylquinolin-1-um-2-yl]-N-propylpropane-1,3-diamine)) was mixed with seawater having 55% salinity at a 1:10,000 ratio. The mixture is incubated for a predetermined period of time, to allow penetration of the indicator through bacterial cell membranes and binding of indicator to, e.g., DNA. The choice of the length of incubation time will vary, and will increase depending upon the degree of sensitivity desired. In the examples which follow, the incubation time was 40 minutes.

After incubation, the sample is pumped to the flow cell, which has been adapted for detection of the indicator via, e.g., fluorescence, such as with an LED light source having an excitation wavelength of 490 nm. This parameter is used because the warm up time is short, and the energy demand is low. Entry and emission of light can be controlled via, e.g., optical filters to minimize interference, and emitted light is measured via spectrometry at 520 nm. It is well known that one can vary LED properties and filters depending upon the nature of the indicator used. Any data secured via the system described herein can be stored on a sensor computer, and is accessible directly or remotely. The skilled artisan will recognize that filters, and LED wavelengths can be changed depending upon the dye used.

The length of operation and number of measurements that may be taken in a given time period are dependent on factors such as the size of the reagent containers.

### Example 1

The following example describes the use of the embodiment described supra. It permitted a set up run for 26 days, with 3 measurements a day before inspection and replacement of reagents were needed.

The biosensor was placed on a flat surface, and connected to a power supply (230 V AC, with live, neutral and earth for power supplies). It should be noted that the system can be adapted for use of solar panels, controls, and batteries.

Further, the sensor was connected to a side stream, having a pressure less than 10 bar. The connection is facilitated by a push in connector, which facilitates connection and replacement of the transport means, which brings water to the system.

In operation, the current system is designed to function at temperatures below 40° C. If temperatures rise above 40° C., incorporated components shut down the system. If components which are not temperature sensitive are used, or cooling means are incorporated into the system, this feature is unnecessary.

The following details the general manner in which the system works; however, variations are possible as will be recognized by the skilled artisan.

The biosensor first measures temperature and humidity to determine if limiting parameters (e.g., temperatures above 40° C., or humidity above 90%) are present. As noted supra, the equipment shuts down if this is the case.

The sensor also provides information on whether any reagents require replenishing.

If the environmental conditions are satisfactory and the necessary quantities of reagent are present, the spectrometer is warmed up, and the system is rinsed with a sample. Following this, both a water sample and a quantity of dye solution using pre-set ratios, are drawn into the syringe. These are mixed via pumping liquids back and forth to the mixing chamber, e.g., three times. After mixing, the sample is incubated, dosed to the flow cell, and fluorescence detected.

Following this, the system is flushed with cleaning agent, distilled water, air, and then distilled water again. The system is then shut down, with automated instructions inputted as to when the process should repeat.

The invention as described herein reduces the time necessary to analyze water samples from weeks, to hours.

### Example 2

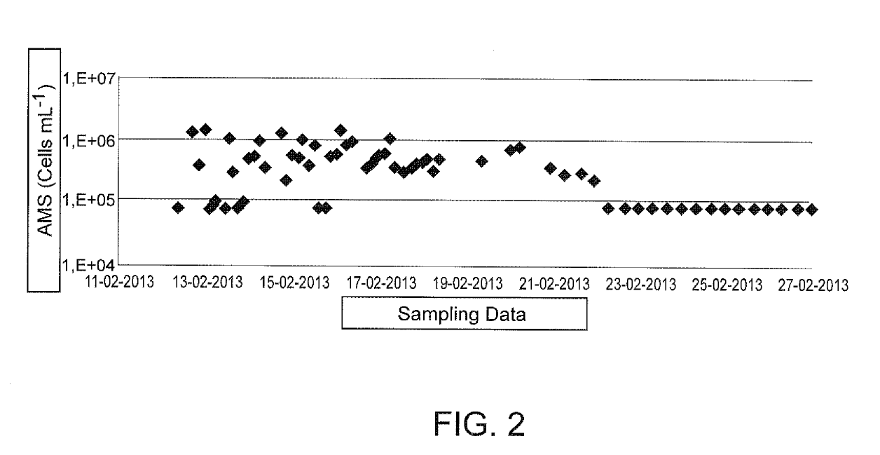

Water supplies were analyzed using the methodology set forth supra. It can be seen, from FIG. 2, that microbial content was high ab initio; however, at the point indicated by the arrow (ten days after measurements begun), a biocide was added, and values were below detection limits. (Note that the data of FIG. 2 represents a correlation between fluorescence signals, and manual counting).

### Example 3

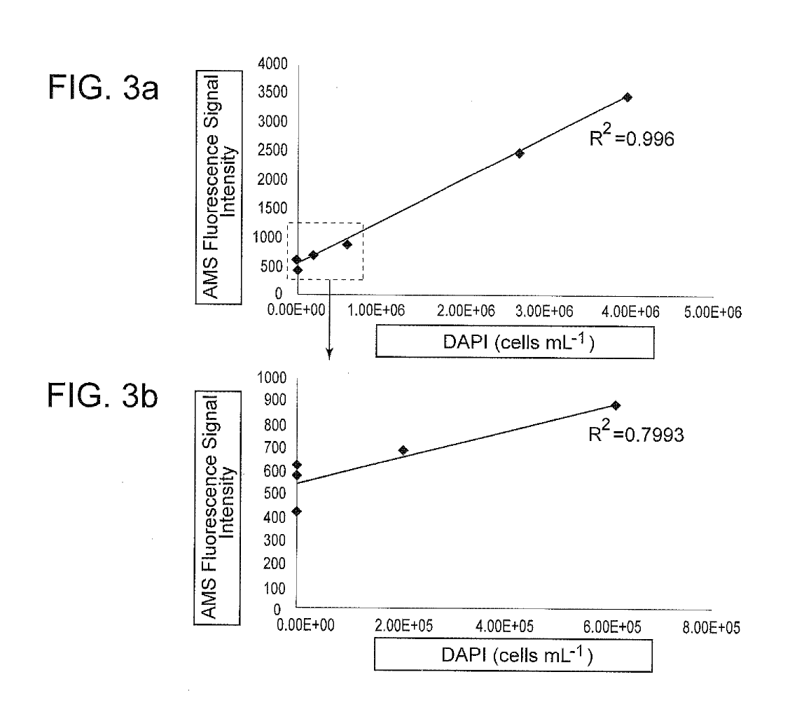

Long-term field-testing of the invention described herein was carried out in Saudi Arabia. FIGS. 3a and 3b present the data, again in terms of correlation of fluorescent staining and manual counting. The linear correlation allows for conversion to cell numbers (cells/ml of seawater). Conversion factors will differ for every system based upon inter alia the chemical composition of the sample, the dye and microbe size.

### Example 4

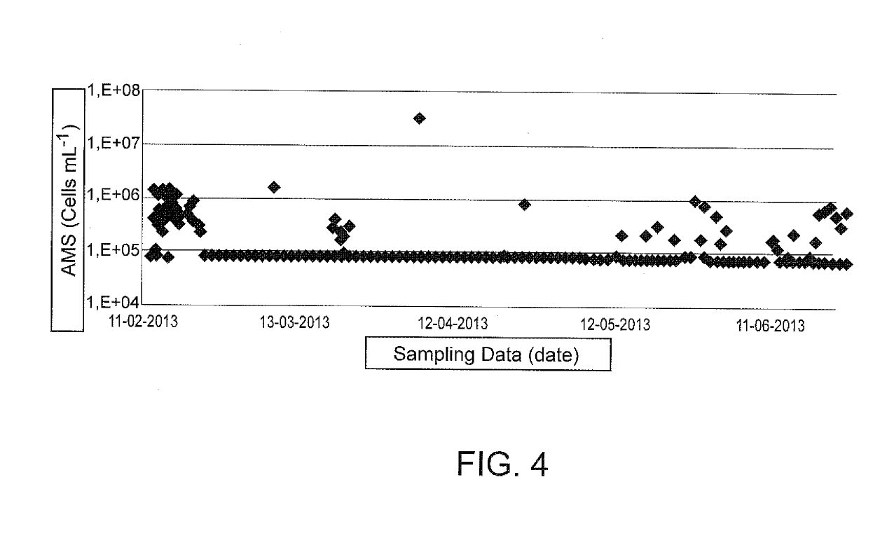

In long-term field tests, microbial content of seawater was measured three times a day, over a 4-month period. The results, shown in FIG. 4, provided valuable information not only about microbial presence but periods where growth rates increased, or decreased. For example, following biocide treatment, there were no detectable microbes, after which growth rates increased.

Other embodiments will be clear to the skilled artisan and need not be reiterated here.

The terms and expression which have been employed are used as terms of description and not of limitation, and there is no intention in the use of such terms and expression of excluding any equivalents of the features shown and described or portions thereof, it being recognized that various modifications are possible within the scope of the invention.

We claim:

1. Apparatus useful in determining presence of microorganisms in a water sample comprising a biosensor connected via a first valve means to a means for providing a water sample to said biosensor, said biosensor further comprising a pressure regulator means and a restrictor means which regulate flow of water to a sample chamber, said sample chamber having a first transport means for transport of a volume of sample to a mixing chamber, an indicator reservoir having a second means for transport of indicator to said mixing chamber, wherein said first and second means for transport have connected thereto an injection valve means for controlling volume of sample and indicator; said mixing chamber having a syringe means connected thereto, said syringe means operating to mix said sample and indicator, means for containing an analysis sample in fluid connection with said mixing chamber, and a means for determining said indicator in said means for containing said analysis sample.

2. The apparatus of claim 1, wherein said means for determining said indicator is a spectrofluorometer.

3. The apparatus of claim 1, further comprising a first reservoir means connected to said biosensor for delivery a quantity of a cleansing agent.

4. The apparatus of claim 3, further comprising a second reservoir means for delivering cleaning water to said biosensor.

5. The apparatus of claim 1, further comprising a waste disposal means connected to said mixing chamber.

6. A method for determining presence of microorganisms in a liquid sample comprising transporting a liquid sample to the mixing chamber of the apparatus of claim 1 together with an indicator sample to form a mixture, incubating said mixture, and determining uptake of said indicator has taken place as an indicator of presence of microorganisms.

7. The method of claim 6, wherein said liquid sample is seawater.

8. The method of claim 6, wherein said indicator is SYBR® green I.
